(19) United States
(12) Patent Application Publication
(10) Pub. No.: US 2025/0057902 A1
(43) Pub. Date: Feb. 20, 2025

(54) U.S. continuation-in-part See p. 2-4

(71) Applicant: Tina Jenkins, Forestville, MD (US)

(72) Inventor: Tina Jenkins, Forestville, MD (US)

(21) Appl. No.: 18/202,917

(22) Filed: Aug. 16, 2023

(51) Int. Cl. A61K 36/185; A23L 13/50; C12G 3/055

(52) U.S. Cl. CPC A61K 36/185; A23L 13/50; C12G 3/055

(57) ABSTRACT

Medical cannabis such as edibles are used as a treatment option for (but is not limited to) a qualifying medical condition or a chronic medical condition which is severe and for which other treatments have been ineffective.

### CROSS-REFERENCE TO RELATED APPLICATION

This application claims the benefit of U.S. Provisional Application Ser. No. 63/075,797, filed on Nov. 27, 2020, entitled “MEDICINAL CANNABIS TEAS,” which is incorporated herein by reference;
This application claims the benefit of U.S. Provisional Application Ser. No. 63/081,885, filed on Nov. 27, 2020, entitled “MEDICINAL CANNABIS BUTTER SPREADS,” which is incorporated herein by reference;
This application claims the benefit of U.S. Provisional Application Ser. No. 63/080,005, filed on Nov. 27, 2020, entitled “MEDICINAL CANNABIS COCOA, CHOCOLATE, AND LEGUME POWDERS,” which is incorporated herein by reference;
This application claims the benefit of U.S. Provisional Application Ser. No. 63/081,899, filed on Nov. 27, 2020, entitled “MEDICINAL CANNABIS BUTTER AND ITS SUBSTITUTES,” which is incorporated herein by reference;
This application claims the benefit of U.S. Provisional Application Ser. No. 63/081,889, filed on Nov. 27, 2020, entitled “MEDICINAL CANNABIS HONEY,” which is incorporated herein by reference;
This application claims the benefit of U.S. Provisional Application Ser. No. 63/081,892, filed on Nov. 27, 2020, entitled “MEDICINAL CANNABIS LIQUID SYRUP(S),” which is incorporated herein by reference;
This application claims the benefit of U.S. Provisional Application Ser. No. 63/080,001, filed on Nov. 27, 2020, entitled “MEDICINAL CANNABIS VITAMIN SUPPLEMENTS,” which is incorporated herein by reference;
This application claims the benefit of U.S. Provisional Application Ser. No. 63/205,364, filed on Nov. 27, 2020, entitled “MEDICINAL CANNABIS ICE CREAM AND ITS SUBSTITUTES,” which is incorporated herein by reference;
This application claims the benefit of U.S. Provisional Application Ser. No. 63/077,574, filed on Nov. 27, 2020, entitled “TO ADD THE DRUG CANNABIS TO ALCOHOLIC BEVERAGES AND ITS SUBSTITUTES,” which is incorporated herein by reference;
This application claims the benefit of U.S. Provisional Application Ser. No. 63/080,011, filed on Nov. 27, 2020, entitled “MEDICINAL CANNABIS COOKING OILS,” which is incorporated herein by reference;
This application claims the benefit of U.S. Provisional Application Ser. No. 63/087,445, filed on Dec. 7, 2020, entitled “MEDICINAL CANNABIS BARBECUE SAUCE(S),” which is incorporated herein by reference;
This application claims the benefit of U.S. Provisional Application Ser. No. 63/205,407, filed on Dec. 7, 2020, entitled “MEDICINAL CANNABIS JUICE BEVERAGES,” which is incorporated herein by reference;
This application claims the benefit of U.S. Provisional Application Ser. No. 63/088,776, filed on Nov. 7, 2020, entitled “TO ADD THE DRUG CANNABIS TO BRAND-NAME SODAS EXCLUDING COCA-COLA,” which is incorporated herein by reference;
This application claims the benefit of U.S. Provisional Application Ser. No. 63/087,452, filed on Dec. 7, 2020, entitled “TO ADD THE DRUG CANNABIS TO MAJOR BRAND-NAME CANDY,” which is incorporated herein by reference;
This application claims the benefit of U.S. Provisional Application Ser. No. 63/081,879, filed on Dec. 7, 2020, entitled “MEDICINAL CANNABIS SMOOTHIES,” which is incorporated herein by reference.

### BACKGROUND OF THE INVENTION

### 1. Field of the Invention

The present inventions are in the field of (but is not limited to) medicine and pertains particularly to edible medical cannabis products.

### 2. Background Art

Edible medical cannabis products are cannabinoid infused products and are intended for human consumption by oral ingestion, in whole or in part, which dissolve or disintegrate in the mouth.

### SUMMARY OF THE INVENTION

Expanding edible medical cannabis products to broaden treatment options for (however is not limited to) eligible consumers with a qualifying medical condition or with a chronic medical condition which is severe and for which other treatments have been ineffective.

### DETAILED DESCRIPTION OF THE INVENTION

To add the drug Cannabis to Tea(s) in general, such as, but is not limited to: Tea bags and Tea beverages, for personal medicinal use to treat medical disorders as well as to expand treatment options. See p. 1 at [0001].

To add the drug Cannabis to Butter Spreads, which is defined as a food paste or spread, such as, but is not limited to: Peanut Butter Spreads, Almond Butter Spreads, Sesame Butter Spreads, and Sun Butter Spreads, for personal medicinal use to treat medical disorders and to extend treatment Options. See p. 1 at [0002].

To add the drug Cannabis to Cocoa Powder(s), Chocolate Powder(s), and Legume Powder(s) commonly used to make hot and cold beverages or used as a food mix, for personal medicinal use to treat medical disorders and to broaden treatment options. See p. 1 at [0003].

To add the drug Cannabis to Butter and its Substitutes, such as, but is not limited to: Butter, Margarine, and Plant-Based Oil Spreads, for personal medicinal use to treat medical disorders and to broaden treatment options. See p. 1 at [0004].

To add the drug Cannabis to Honey in general, which is defined as: A food substance made by honey bees and some related insects, for personal medicinal use to treat medical disorders as well as to expand treatment options. See p. 1 at [0005].

To add the drug Cannabis to Liquid Syrup(s) in general, such as, but is not limited to: Chocolate Syrup(s), Breakfast Syrup(s), and Medicinal Syrup(s), for personal medicinal use to treat medical disorders and to broaden treatment options. See p. 1 at [0006].

To add the drug Cannabis to Vitamin Supplements in general, such as, but is not limited to: Multivitamin Supplements and Single Vitamin Supplements, for personal medicinal use to treat medical disorders while providing nourishment to the body, as well as for a more extensive treatment option selection. See p. 2 at [0007].

To add the drug Cannabis to Ice Cream(s) and its Substitutes, such as, but is not limited to: Personal-size Ice Cream(s), Frozen Yogurt(s), Frozen Custard(s), Sherbet(s), Sorbet(s), Gelato(s), Granita(s), Italian Ice(s), and Popsicle(s), for personal medicinal use to treat medical disorders as well as to expand treatment options. See p. 2 at [0008].

To add the drug Cannabis to Alcoholic Beverages and its Substitutes, such as, but is not limited to: Beer, and its substitutes such as beer beverages without intoxicating agents, for personal medicinal use to treat medical disorders and to broaden treatment options. See p. 2 at [0009].

To add the drug Cannabis to Cooking Oils in general, which is defined as: Plant, Animal, or Synthetic Fat used in frying, baking, and other types of cooking. It is also used in food preparation and flavoring not involving heat, such as salad dressings and bread dippings like bread dips, and may be called edible oil, for personal medicinal use to treat medical disorders. See p. 2 at [0010].

To add the drug Cannabis to Barbecue Sauce(s) in general, which is defined as: A sauce used as a Marinade, Basting, Condiment, or Topping for food, for personal medicinal use to treat medical disorders and to broaden treatment options. See p. 2 at [0011].

To add the drug cannabis to Juice Beverages in general, such as, but is not limited to: Lemonade, Limeade, and Fruit Punch, for personal medicinal use to treat medical disorders and to broaden treatment options. See p. 2 at [0012].

To add the drug Cannabis to Brand-Name Sodas excluding Coca-Cola, such as, but is not limited to: Ginger Ale and Pepsi Cola, for personal medicinal use the treat medical disorders and for a more extensive medicinal selection. See p. 2 at [0013].

To add the drug Cannabis to Major Brand-Name Candy, such as, but is not limited to: Candy Bars, Boxed Chocolates, and Lollipops, for personal medicinal use to treat medical disorders as well as to expand treatment options. See p. 2-3 at [0014].

To add the drug Cannabis to Smoothie Beverages in general, such as, but is not limited to: Freshly made smoothie beverages and/or grab-and-go smoothie beverages, for personal medicinal use to treat medical disorders and to broaden treatment options. See p. 3 at [0015].

What is claimed is:

1. Local first-filed patent applications followed within 12 months by multiple foreign applications claiming priority under the Paris Convention of 1883.

2. Expansion of edible medical cannabis products.

3. Broadening treatment options.
